(19) United States
(12) Patent Application Publication
(10) Pub. No.: US 2025/0282806 A1
(43) Pub. Date: Sep. 11, 2025

(54) CHEMICAL SYNTHESIS METHOD FOR DISACCHARIDE FRAGMENT OF VIBRIO VULNIFICUS BIOTYPE 2 SEROVAR A O-ANTIGEN

(71) Applicant: Jiangnan University, Wuxi (CN)

(72) Inventors: Jian YIN, Wuxi (CN); Jing HU, Wuxi (CN); Shengyong ZHU, Wuxi (CN); Chunjun QIN, Wuxi (CN)

(21) Appl. No.: 19/216,855

(22) Filed: May 23, 2025

Related U.S. Application Data
(63) Continuation of application No. PCT/CN2024/127387, filed on Oct. 25, 2024.

(30) Foreign Application Priority Data
May 14, 2024 (CN) 2024105925905

(51) Int. Cl. C07H 1/02; A61K 31/7016; A61K 39/02; A61K 45/06; C07H 3/04

(52) U.S. Cl. CPC C07H 1/02; A61K 31/7016; A61K 39/107; A61K 45/06; C07H 3/04

(57) ABSTRACT

The disclosure provides a chemical synthesis method for a disaccharide fragment of a Vibrio vulnificus biotype 2 serovar A O-antigen, belonging to the chemical field. The disclosure utilizes D-glucose and L-galactose as raw materials to prepare two types of glycosylated building blocks, and designs an efficient synthesis route for constructing the disaccharide fragment. By optimizing protecting groups and optimizing the time of introducing modifying groups, the preparation of the target disaccharide is successfully completed. The raw materials for preparing the disaccharide in the disclosure are readily available, the preparation method is simple and easy to repeat, and will have a good application prospect in the development of new drugs and vaccines for V. vulnificus.

### TECHNICAL FIELD

The disclosure relates to a chemical synthesis method for a disaccharide fragment of a V. vulnificus biotype 2 serovar A O-antigen, and in particular to a heteromodified polyamino oligosaccharide with an amino linker assembled at a reducing end, belonging to the chemical field.

### BACKGROUND

V. vulnificus is a Gram negative bacterium widely distributed in seawater and seafood (C. Baker-Austin et al., Trends Microbiol. 2020, 28, 81-82), and is one of the three major Vibrio species threatening human health (C. Baker-Austin et al., Nat. Rev. Dis. Primers. 2018, 4, 1-19). V. vulnificus infection may lead to severe diseases such as sepsis and gangrene, with a mortality rate of up to 50% (WHO, Food & Agriculture Org., 2021). The harm of V. vulnificus is attributed to various pathogenic factors, including capsular polysaccharide (CPS), lipopolysaccharide (LPS), cilia, flagella, cytotoxin, enzyme, hemolysin, and cytolysin. In clinical practice, V. vulnificus can be classified into three biotypes (1-3) (O. James D et al., In The biology of vibrios. 2006, 349-366). Up to now, about 40 highly virulent pathogenic strains have been discovered in clinical practice. Most human infections with V. vulnificus are related to biotype 1 and biotype 2 strains, with biotype 2 having a higher mortality rate. It is worth noting that the biotype 2 strain is the only zoonotic strain (C. Amaro et al., In Vibrio spp. Infections. 2023, 175-194; F. Roig et al., Appl. Environ. Microbiol. 2023, 18, 373-383). In addition, biotype 2 strains are further divided into serovar A and E (C. Amaro et al., Appl. Environ. Microbiol. 1996, 62, 1454-1457; E. Sanjuán et al., Appl. Environ. Microbiol. 2004, 70, 7024-7032).

Usually, V. vulnificus infections may be treated with antibiotics combined with surgeries, and amputation may be required if necessary (K. Coerdt et al., A. Cutis. 2021, 107, E12-E17). Recent reports indicate that the effectiveness of using antibiotics to control V. vulnificus infections is very limited, repeated outbreaks of the disease are encountered, and multiple antibiotic-resistant strains have been isolated. Therefore, there is an urgent need to develop relevant vaccines for V. vulnificus (F. Roig et al., Appl. Environ. Microbiol. 2023, 18, 373-383). At present, research on V. vulnificus vaccines is mainly limited to fish (Q. Ji, et al., Immunol. Lett. 2020, 226, 46-54). The existing V. vulnificus veterinary vaccines are based on intact organisms and composed of live attenuated or inactivated organisms (S. Lee, et al., Infect. Immun. 2006, 74, 694-702). In recent years, some new V. vulnificus vaccines have emerged, including DNA vaccines and vector vaccines, but have not yet been introduced to the market. Researchers have employed various methods to develop human V. vulnificus vaccines, including whole cell and attenuated live vaccines (Q. Ji et al., Aquaculture. 2022, 560, 738560). However, there is still controversy over the long-term preventive efficacy on V. vulnificus, and as of now, there is no effective vaccine available for human infections with V. vulnificus (. Baker-Austin et al., Nat. Rev. Dis. Primers. 2018, 4, 1-19). On the other hand, since many antigenic determinants in inactivated vaccines may induce non-specific immune responses, subunit vaccines based on surface polysaccharides, membrane proteins and the like of V. vulnificus are considered to provide potential and more effective immune protection (G. Pettis et al., Int. J. Mol. Sci. 2020, 21, 3259). Lipopolysaccharide O-antigens of V. vulnificus biotypes 1 and 2 exhibit strong structural specificity, making them potential immune targets for the development of V. vulnificus vaccines (C. Baker-Austin et al., Trends Microbiol. 2020, 28, 81-82). Zhang Han et al. reported that the combination of V. vulnificus biotype 1 (MO6-24 and BO62316) O-antigen and carrier protein CRM197 can effectively induce T-cell dependent immune responses in mice (H. Zhang et al., JACS Au. 2021, 2, 97-108.). Collado et al. measured antibody titers and bactericidal/bacteriostatic activity in eel mucus and serum in their study, demonstrating that the V. vulnificus biotype 2 O-antigen is a potential immune target (R. Collado et al., Dis. Aquat. Org. 2000, 43, 91-101). However, the O-antigen obtained through biological pathways often exhibit structural heterogeneity, with residual lipid A and other cellular impurities that may reduce the safety and efficacy of candidate vaccines (P. H. Seeberger, Chem. Rev. 2021, 121, 3598-3626; C. Qin et al., Curr. Opin. Chem. Biol. 2024, 78, 102424). Therefore, the preparation of clear oligosaccharides corresponding to the O-antigen through chemical methods has become a valuable tool for the continuous development of saccharide vaccines.

A V. vulnificus biotype 2 serovar A O-antigen includes a characteristic disaccharide fragment [→4)-α-L-GalpNAmA-(1→3)-α-D-QuipNAc-(1→], which has a rare acetamidino group modification (A. Shashkov et al., Carbohydr. Res. 2009, 344, 2005-2006.). The disaccharide fragment with this structure can serve as a potential target for immune related applications (C. Qin et al., Chin. J. Nat. Med. 2022, 20, 401-420; G. Tian et al., Molecules. 2023, 28, 7112.). Based on chemical synthesis, an oligosaccharide with a single structure and high purity can be obtained, which is beneficial for promoting the study on a structure-activity relationship. So far, people have been committed to synthesizing rare deoxyaminoglucoses, assembling complex polysaccharides, and installing rare modifying groups. Nevertheless, rare L-aminogalacturonic acid, two 1,2-cis glycosidic bonds, and an acetamidino group make the chemical synthesis of this disaccharide still a challenge (J. Harjani et al., 2011, 76, 1683-1691). In addition, selecting to assemble an orthogonal linker at a reducing end can facilitate conjugation or fixation in biological research. Considering that the active part of the linker may influence the synthesis efficiency of the disaccharide, it is necessary to evaluate the practicality of different linkers. Therefore, the chemical synthesis of this structure requires a holistic route design, including the selection of protecting groups, the selection of time for introducing modifying groups, the efficiency and selectivity of glycosylation reaction, and other key links. This synthesis will play a key role in evaluating the activity of the disaccharide fragment of the V. vulnificus biotype 2 serovar A O-antigen and the development of saccharide vaccines.

### SUMMARY

The disclosure relates to a chemical synthesis method for a disaccharide fragment assembled with a linker of a V. vulnificus biotype 2 serovar A O-antigen. A chemical structural formula of the disaccharide fragment is as expressed by general formula I:

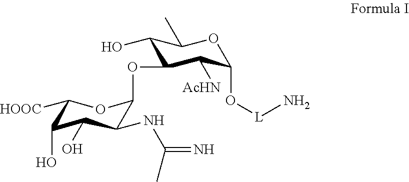

In the disclosure, a linker L may be a chain structure with 2-40 carbon atoms (including carbon atoms in side chains) and containing 0-6 heteroatoms.

In the disclosure, when the length of a main chain of the linker is 4-8 atoms, the main chain may include 1, 2, or 3 heteroatoms (O, N, and S). When the length of the main chain of the linker is 9-14 atoms, the main chain may include 1, 2, 3, 4, 5, or 6 heteroatoms (O, N, and S).

In the disclosure, the linker -L- may be a fully or partially fluorine-substituted ring structure. The linker -L- may contain a three-membered, four-membered, five-membered or six-membered saturated carbocyclic ring; it may also contain a five-membered unsaturated carbocyclic ring (non-aromatic ring); it may also contain a four-membered, five-membered or six-membered saturated oxygen heterocyclic ring; it may also contain a four-membered, five-membered or six-membered saturated nitrogen heterocyclic ring; and it may also contain a six-membered aromatic carbocyclic ring.

In the disclosure, the linker -L- may also contain an amide bond and/or a carbamido group.

In the disclosure, the above linker -L- may also contain one or more substituent groups, and the substituent groups may include: —F, —Cl, —CH₃, —C₂H₅, —C₃H₇, —C₅H₉, —C₆H₁₃, —OCH₃, —OC₂H₅, —CH₂F, —CHF₂, —CF₃, —C(O)—NH₂, —SCH₃, —SC₂H₅, —NHC(O)CH₃, —N(CH₃)₂, and —N(C₂H₅)₂.

In the disclosure, the synthesized saccharide chain structure contains basic (acetamidino) and acidic (carboxyl) groups, which may form corresponding salts with organic or inorganic acids or bases. The acids that may be used for salt formation include hydrochloric acid, hydrobromic acid, sulfuric acid, phosphoric acid, acetic acid, citric acid, oxalic acid, lactic acid, malonic acid, salicylic acid, para-aminosalicylic acid, malic acid, fumaric acid, succinic acid, ascorbic acid, maleic acid, sulfonic acid, phosphonic acid, perchloric acid, nitric acid, propionic acid, methanesulfonic acid, ethanesulfonic acid, mandelic acid, picric acid, adipic acid, gluconic acid, tartaric acid, nitrous acid, hydroxymaleic acid, pyruvic acid, phenylacetic acid, benzoic acid, para-aminobenzoic acid, para-hydroxybenzoic acid, hydroxyethyl sulfonic acid, ethylene sulfonic acid, para-toluenesulfonic acid, naphthalene sulfonic acid, para-aminobenzenesulfonic acid, camphorsulfonic acid, ortho-methylmandelic acid, hydroxybenzenesulfonic acid, ortho-toluene tartaric acid, tartronic acid, aminonaphthalene sulfonic acid, and other mineral acids or carboxylic acid substances. Inorganic or organic bases that can be used for forming a salt are sodium hydroxide, potassium hydroxide, aqueous ammonia, tetraalkylammonium hydroxide, lysine, arginine and the like.

In the disclosure, the synthesized saccharide chain structure contains both basic (acetamidino) and acidic (carboxyl) groups, and intramolecular protons, i.e., protons of acidic group, may be transferred to the basic group. General formula I may be an amphoteric molecule containing —O— and —NH₃⁺.

In an implementation of the disclosure, the chemical synthesis method for the disaccharide fragment assembled with the linker of the V. vulnificus biotype 2 serovar A O-antigen expressed by general formula (I) above uses the following two monosaccharide building blocks 1 (D-quinovosamine) and 2 (L-aminogalacturonic acid) and a linker 3 as raw materials:

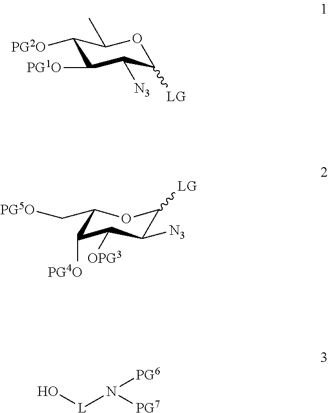

where
PG¹ is a hydroxyl protecting group which may be selected from acetyl (Ac), benzoyl (Bz), levulinoyl (Lev), pivaloyl (Piv), allyloxycarbonyl (Alloc), chloroacetyl (ClAc), dichloroacetyl (DCA), trichloroacetyl (TCA), 2-naphthylmethyl (Nap), para-methoxybenzyl (PMB), tert-butyldimethylsilyl (TBDMS), tert-butyldiphenylsilyl (TBDPS), and triethylsilyl (TES);
PG² is a hydroxyl protecting group which may be 2-naphthylmethyl (Nap) or benzyl (Bn);
PG³ is a hydroxyl protecting group which may be 2-naphthylmethyl, benzyl, acetyl, levulinoyl, benzoyl, chloroacetyl, dichloroacetyl, trichloroacetyl, pivaloyl, allyloxycarbonyl, benzyl, 2-naphthylmethyl, para-methoxybenzyl, tert-butyldimethylsilyl, tert-butyldiphenylsilyl, and triethylsilyl;
PG⁴ and PG⁵ are hydroxyl protecting groups which may be benzylidene acetal (PhCH) or isopropylidene ketal ((CH₃)₂CH);
PG⁶ and PG⁷ are amino protecting groups which may be benzyl (Bn) or benzyloxycarbonyl (Cbz); and
LG is a leaving group used for a glycosylation reaction which may be bromine, fluorine, ethylthio, para-tolylthio, phenylthio, trichloroacetimidate, N-phenyl trifluoroacetimidate, or dibutyl phosphate; and
the chemical synthesis method sequentially includes the following steps:
reaction A (glycosidation reaction): enabling the monosaccharide building block 1 to experience a glycosidation reaction with the linker 3;
reaction B (reduction and acetylation of azido group): after completing the glycosidation reaction, performing reduction and acetylation on an azido group at position 2 in the structure;
reaction C (deprotection on position 3 in quinovosamine): after completing the reduction and acetylation of the azido group, performing deprotection on position 3 to remove the protecting group PG¹ to obtain a D-quinovosamine receptor used for assembling a disaccharide, and then enabling the D-quinovosamine receptor to experience a glycosidation reaction with the monosaccharide building block 2 to obtain an initial disaccharide;
reaction D (deprotection of positions 4 and 6 in galactosamine): performing deprotection on positions 4 and 6 in the monosaccharide building block 2 in the obtained initial disaccharide to remove the protecting groups PG⁴ and PG⁵;
reaction E (oxidization of primary hydroxyl group to carboxyl group): after completing the deprotection, oxidizing a primary hydroxyl group at position 6 in the monosaccharide building block 2 to a carboxyl group;
reaction F (protection of carboxyl group): subsequently performing protection on the carboxyl group after oxidization;
reaction G (reduction of azido group to amino group): after the carboxyl group is protected, reducing the azido group at position 2 in the monosaccharide building block 2 to an amino group;
reaction H (modification of amino group to acetamidino group): after reducing to the amino group, constructing an acetamidino group through a modifying reagent; and finally
reaction I (global deprotection): performing global deprotection to remove the protecting group PG² in the monosaccharide building block 1, the protecting group of the carboxyl group and the protecting group PG³ in the monosaccharide building block 2, and the protecting groups PG⁶ and PG⁷ in the linker 3 to obtain the disaccharide fragment assembled with the linker of the V. vulnificus biotype 2 serovar A O-antigen as expressed by general formula (I).

In an implementation of the disclosure, a route of the above synthesis process is as follows:

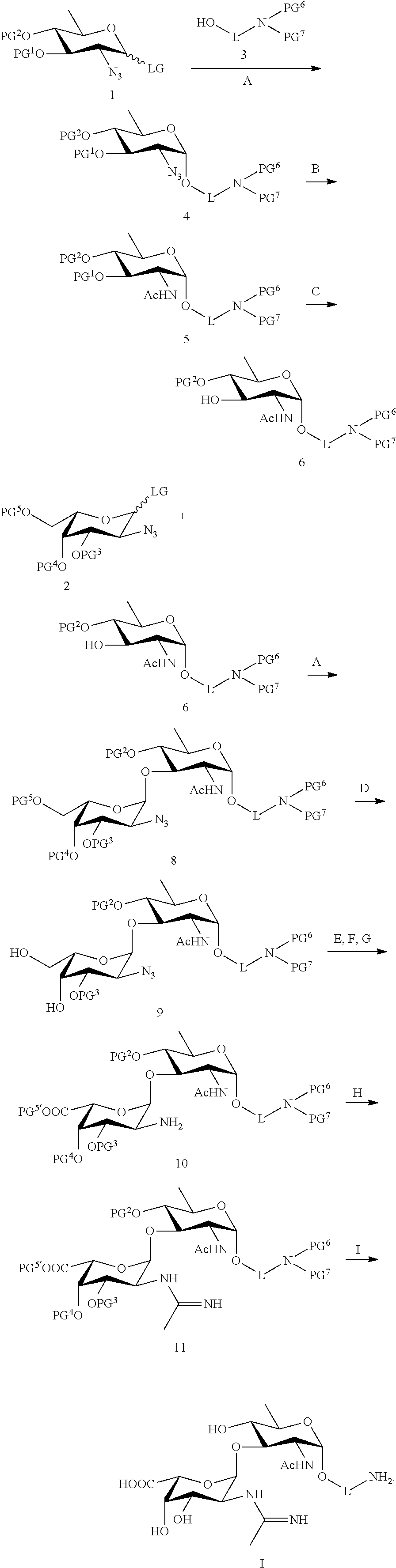

In an implementation of the disclosure, 1) in reaction A (glycosidation reaction), in a case that the leaving group used in the glycosidation reaction is ethylthio, para-tolylthio, or phenylthio, the activator in the glycosidation reaction may be any one or more selected from methyl trifluoromethanesulfonate (TfOMe), dimethylmethylthiosulfonium trifluoromethanesulfonate (DMTST), trifluoromethanesulfonic acid (TfOH), and trimethylsilyl trifluoromethanesulfonate (TMSOTf), and the reaction temperature may range from −40° C. to room temperature;
in a case that the leaving group is fluorine, the activator in the glycosidation reaction may be silver perchlorate (AgClO₄), titanium tetrafluoride (TiF₄), trifluoromethanesulfonic anhydride (Tf₂O), or the like, and the reaction temperature may range from −40° C. to room temperature;
in a case that the leaving group is bromine, the activator in the glycosidation reaction may be silver perchlorate (AgClO₄) or silver trifluoromethanesulfonate, and the reaction temperature may range from −40° C. to room temperature; and
in a case that the leaving group is trichloroacetimidate, N-phenyl trifluoroacetimidate, or dibutyl phosphate, the activator in the glycosidation reaction may be a boron trifluoride-ether complex (BF₃·OEt₂), trimethylsilyl trifluoromethanesulfonate (TMSOTf), or silver trifluoromethanesulfonate (AgOTf), and the reaction temperature may range from −40° C. to room temperature.

A molecular sieve used for water removal in the glycosidation reaction may be a 4 Å molecular sieve or a 3 Å molecular sieve.

In an implementation of the disclosure, 2) in reaction B (reduction and acetylation of azido group),
reduction and acetylation of the azido group may be completed directly through a thioacetic acid (AcSH)/pyridine method, or may be completed by reducing the azido group to an amino group and then performing acetylation. A method of reducing the azido group to the amino group may be performed through trimethylphosphine (PMe₃)/water, triphenylphosphine (PPh₃)/water, 1,3-dimercaptopropane/triethylamine, sodium borohydride (NaBH₄)/nickel dichloride (NiCl₂), tin dichloride (SnCl₂)/phenylthiol (PhSH)/triethylamine, zinc/copper/acetic acid, Lindlar catalyst/hydrogen, or the like.

A method of acetylating the amino group may be performed through acetic anhydride (Ac₂O)/methanol, acetic anhydride/pyridine, acetyl chloride (AcCl)/triethylamine, or the like.

In an implementation of the disclosure, 3) in reaction C (deprotection on position 3 in quinovosamine),
in a case that the hydroxyl protecting group is acetyl (Ac), benzoyl (Bz), chloroacetyl (ClAc), dichloroacetyl (DCA), trichloroacetyl (TCA) or pivaloyl (Piv), a deprotection condition used may be sodium methoxide/methanol, potassium hydroxide/methanol, sodium hydroxide/methanol, or the like;
in a case that the hydroxyl protecting group is levulinyl (Lev), the deprotection condition used may be hydrazine acetate/pyridine or the like;
in a case that the hydroxyl protecting group is allyloxycarbonyl (Alloc), the deprotection condition used may be palladium diacetate (Pd(OAc)₂)/diethylamine or the like;
in a case that the hydroxyl protecting group is 2-naphthylmethyl (Nap) or para-methoxybenzyl (PMB), the deprotection condition used may be 2,3-dichloro-5,6-dicyano-1, 4-benzoquinone (DDQ)/water, ceric ammonium nitrate (CAN)/water, or the like; and
in a case that the hydroxyl protecting group is tert-butyldimethylsilyl (TBDMS), tert-butyldiphenylsilyl (TBDPS), or triethylsilyl (TES), the deprotection condition used may be tetrabutylammonium fluoride (TBAF), hydrofluoric acid, or the like.

In an implementation of the disclosure, 4) in reaction D (deprotection on positions 4 and 6 in galactosamine),
in a case that the hydroxyl protecting group at positions 4 and 6 in galactosamine is benzylidene acetal (PhCH), a deprotection condition used may be acetic acid aqueous solution, sulfuric acid aqueous solution, trifluoroacetic acid aqueous solution, Pd(OH)₂, Pd/C catalytic hydrogenolysis, or the like; and
in a case that the hydroxyl protecting group at positions 4 and 6 in galactosamine is benzylidene acetal or isopropylidene ketal ((CH₃)₂CH), the deprotection condition used may be acetic acid methanol solution, sulfuric acid methanol solution, trifluoroacetic acid methanol solution, methanol solution of all proton acids and Lewis acids, silica gel or acidic ion exchange resin, or the like.

In an implementation of the disclosure, 5) in reaction E (oxidization of primary hydroxyl group to carboxyl group),
a method of oxidizing the primary hydroxyl group to the carboxyl group may be performed through treatment by using any one or more of Jones reagent (CrO₃/H₂SO₄), Collins reagent (CrO₃/py), pyridinium chlorochromate, and pyridinium dichromate;
it may also be performed based on dimethyl sulfoxide (DMSO), where the activator may be one or more of dicyclohexyl carbodiimide (DCC), acetic anhydride, trifluoroacetic anhydride, phosphorus pentoxide, pyridine/sulfur trioxide, and oxalyl chloride;
it may also be performed through treatment by using a transition metal in oxygen; and
it may also be performed through treatment by using (2,2,6,6-tetramethylpiperidine oxide) TEMPO and nitrogen-oxygen radical active substances thereof.

In an implementation of the disclosure, 6) in reaction F (protection of carboxyl group), the protecting group of the carboxyl group of galacturonic acid (PG⁵′) may be methyl, tert-butyl, allyl, triphenylmethyl, diphenylmethyl, tert-butyldimethylsilyl (TBS), propargyl, benzyl (Bn), para-methoxybenzyl (PMB), or the like.

In an implementation of the disclosure, 7) in reaction G (reduction of azido group to amino group),
A method of reducing the azido group to the amino group may be performed through trimethylphosphine (PMe₃)/water, triphenylphosphine (PPh₃)/water, 1,3-dimercaptopropane/triethylamine, sodium borohydride (NaBH₄)/nickel dichloride (NiCl₂), tin dichloride (SnCl₂)/phenylthiol (PhSH)/triethylamine, zinc/copper/acetic acid, Lindlar catalyst/hydrogen, or the like.

In an implementation of the disclosure, 8) in reaction H (modification of amino group to acetamidino group),
a method of modifying the amino group to the acetamidino group may be performed by using aryl thioacetimidate hydrohalide or alkyl thioacetimidate hydrohalide under treatment of an alkali; aryl may be benzyl, naphthylmethyl, or the like; alkyl may be methyl, ethyl, or the like; halogen acid may be hydrochloric acid, hydrobromic acid, or the like; the alkali used may be pyridine, triethylamine, diisopropylethylamine, 1,8-diazabicyclo[5.4.0]undec-7-ene (DBU), potassium carbonate, sodium carbonate, or the like.

The method of modifying the amino group to the acetamidino group may be performed by using alkyl acetimidate hydrohalide under treatment of an alkali; alkyl may be ethyl, trifluoroethyl, trichloroethyl, or the like; halogen acid may be hydrochloric acid, hydrobromic acid, or the like; the alkali used may be pyridine, triethylamine, diisopropylethylamine, 1,8-diazabicyclo[5.4.0]undec-7-ene (DBU), potassium carbonate, sodium carbonate, or the like.

In an implementation of the disclosure, 9) in reaction I (global deprotection), global deprotection through catalytic hydrogenation may be achieved through a reaction at room temperature under a catalytic condition by feeding hydrogen. A catalyst used for catalytic hydrogenation may be a 10% palladium/carbon catalyst, palladium hydroxide, or the like; a solvent used for the reaction may be water/methanol/dichloromethane/acetic acid mixed solution, water/tert-butanol/dichloromethane mixed solution, or the like.

In a case that the protecting group of the carboxyl group is methyl, the protecting group of the carboxyl group needs to be firstly removed through sodium hydroxide aqueous solution, and then global deprotection is performed through catalytic hydrogenation;
in a case that the protecting group of the carboxyl group is tert-butyl, tert-butyldimethylsilyl (TBS), or para-methoxybenzyl (PMB), the protecting group of the carboxyl group needs to be firstly removed through formic acid, acetic acid, or trifluoroacetic acid, and then global deprotection is performed through catalytic hydrogenation;
in a case that the protecting group of the carboxyl group is allyl, the protecting group of the carboxyl group needs to be firstly removed through palladium acetate, sodium 2-methylhexanoate, or triphenylphosphine in acetone, and then global deprotection is performed through catalytic hydrogenation;
in a case that the protecting group of the carboxyl group is triphenylmethyl, the protecting group of the carboxyl group needs to be firstly removed through methanol or water/dioxane, and then global deprotection is performed through catalytic hydrogenation; and
in a case that the protecting group of the carboxyl group is diphenylmethyl or benzyl (Bn), global deprotection is directly performed through catalytic hydrogenation.

In the chemical synthesis method for the disaccharide fragment modified with the linker of the V. vulnificus biotype 2 serovar A O-antigen provided in the disclosure, in a case that the monosaccharide building block 1 (quinovose) is used as a glycosyl donor, the amino group at position 2 may be protected by an azido group to facilitate the generation of a 1,2-cis-α-glycosidic bond.

In the chemical synthesis method for the disaccharide fragment modified with the linker of the V. vulnificus biotype 2 serovar A O-antigen provided in the disclosure, in a case that the monosaccharide building block 2 (galactose) is used as a glycosyl donor, the amino group at position 2 may be protected by an azido group to facilitate the generation of a 1,2-cis-α-glycosidic bond.

In the chemical synthesis method for the disaccharide fragment modified with the linker of the V. vulnificus biotype 2 serovar A O-antigen provided in the disclosure, the acetamido group at position 2 in the structure of the monosaccharide building block 1 (quinovose) may be introduced immediately after the glycosylation reaction of the monosaccharide building block 1 (quinovose) is completed, so as to avoid the situation of hindering the subsequent introduction of the monosaccharide building block.

In the chemical synthesis method for the disaccharide fragment modified with the linker of the V. vulnificus biotype 2 serovar A O-antigen provided in the disclosure, the carboxyl group at position 6 in the structure of the monosaccharide building block 2 (galactose) may be introduced immediately after the construction of a disaccharide skeleton is completed, so as to avoid the situation of carrying the carboxyl group and affecting the glycosylation efficiency.

In the chemical synthesis method for the disaccharide fragment modified with the linker of the V. vulnificus biotype 2 serovar A O-antigen provided in the disclosure, compared with a disaccharide 17* assembled with a sulfhydryl linker and oxidized in a monosaccharide stage, the glycosylation efficiency is significantly improved (FIG. 11). The total yield of synthesizing a disaccharide-like skeleton from L-galactose as a raw material increases from 4.5% to 15.8%.

The disclosure provides a disaccharide fragment assembled with a linker of a V. vulnificus biotype 2 serovar A O-antigen prepared by adopting the chemical synthesis method described above.

The disclosure further provides a V. vulnificus vaccine, which contains the disaccharide fragment assembled with the linker of the V. vulnificus biotype 2 serovar A O-antigen described above.

The disclosure further provides a drug for treating a disease caused by V. vulnificus infection, which contains the disaccharide fragment assembled with the linker of the V. vulnificus biotype 2 serovar A O-antigen described above.

In an implementation of the disclosure, the drug composition further contains a pharmaceutical auxiliary material.

In an implementation of the disclosure, the drug composition further contains a pharmaceutically acceptable diluent, an adjuvant, a carrier, or an excipient.

### Beneficial Effects

The disclosure achieves the chemical synthesis of the disaccharide fragment modified with the linker of the V. vulnificus biotype 2 serovar A O-antigen for the first time. In addition, by introducing the corresponding linker at the reducing end of the saccharide structure, it provides a basis for synthesizing oligosaccharide structure coupled carrier molecules or immobilizing them on corresponding matrices, thus facilitating the study on the biological effect of the original saccharide structure of the V. vulnificus biotype 2 serovar A O-antigen and the development of saccharide vaccines.

### BRIEF DESCRIPTION OF FIGURES

FIG. 1 shows a disaccharide fragment of a V. vulnificus biotype 2 serovar A O-antigen.

FIG. 2 shows a compound expressed by general formula I.

FIG. 3 shows compounds of monosaccharide building blocks 1 and 2 and a linker 3.

FIG. 4 shows a synthesis reaction equation of α-D-quinovopyranose 6.

FIG. 5 shows a synthesis reaction equation of a disaccharide fragment I of a V. vulnificus biotype 2 serovar A O-antigen.

FIG. 6 shows a chemical synthesis reaction equation of a compound 3*.

FIG. 7 shows a chemical synthesis reaction equation of a compound 6*.

FIG. 8 shows a chemical synthesis reaction equation of a compound 12*.

FIG. 9 shows a chemical synthesis reaction equation of a compound 15*.

FIG. 10 shows a chemical synthesis reaction equation of a compound 16*.

FIG. 11 shows a chemical synthesis reaction equation of a compound 17*.

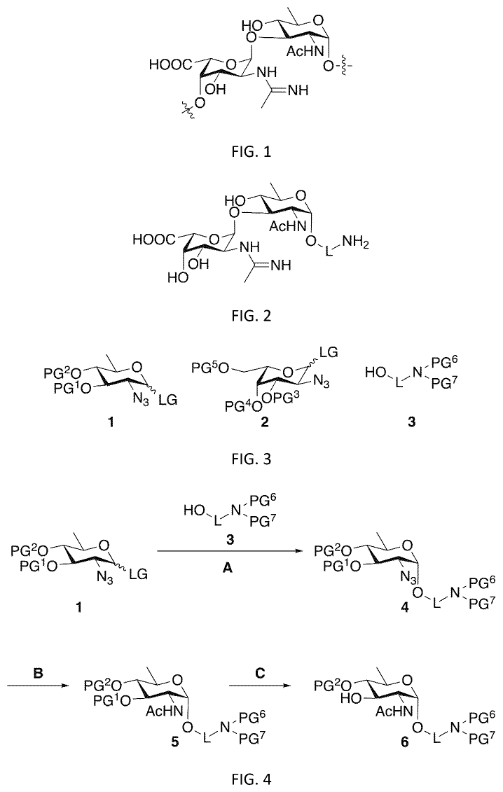

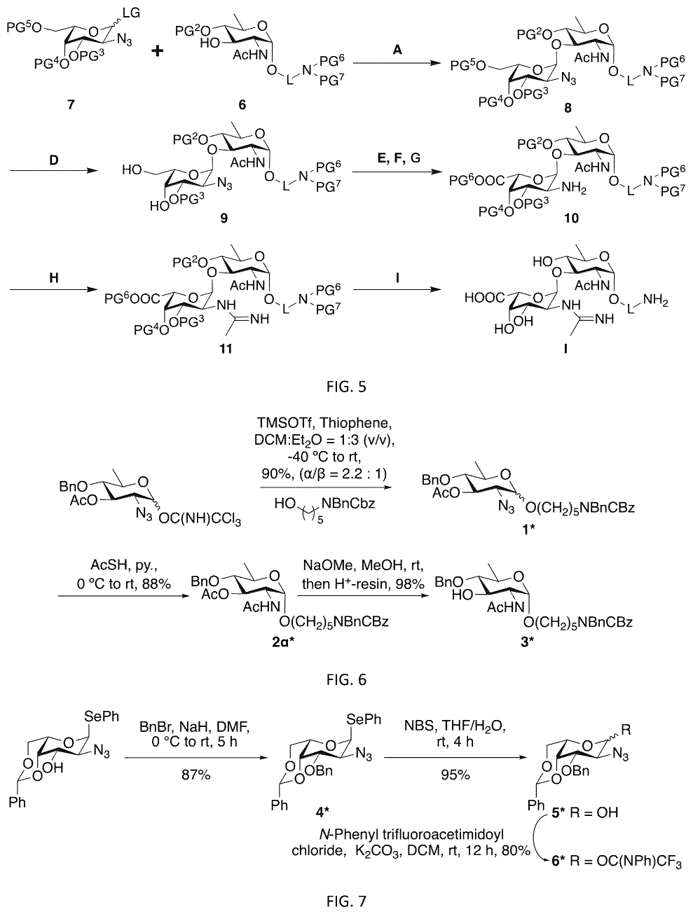

### DETAILED DESCRIPTION

Commercially available reagents used in experiments were directly used without treatment. An anhydrous solvent used in reaction was prepared by an MBraun MB-SPS 800 solvent drying system. Solvents used in silica gel column chromatography were all analytically pure and used after distillation under reduced pressure. A silica gel plate used for Thin Layer Chromatography (TLC) was a glass-based or aluminum-box-based silica gel plate prepared by using 60-F254 silica gel. A visualization reagent for thin layer chromatography was a saccharide visualization reagent (90.1% (v/v) 3-methoxyphenol and 2.5% (v/v) sulfuric acid ethanol solution), or a CAM visualization reagent (5% (w/v) ammonium molybdate, 1% (w/v) cerium (II) sulfate, and 10% (v/v) sulfuric acid aqueous solution), or a ninhydrin visualization reagent (1.5% (w/v) ninhydrin and 3% (v/v) acetic acid n-butanol solution). Silica gel used for normal-phase silica gel column chromatography was 200-mesh to 300-mesh silica gel. A filler used for molecular exclusion chromatography is dextran gel SephadeX®LH-20 (GE Healthcare).

The yield of each reaction step was calculated separately, and the yield was calculated according to (amount of target product/amount of raw material)×100%. The structure of each product was identified by nuclear magnetic spectroscopy, infrared spectroscopy, optical rotation and high-resolution mass spectrometry. The purity of each product was analyzed by nuclear magnetic spectroscopy and high performance liquid chromatography. Hydrogen spectra, carbon spectra, and two-dimensional nuclear magnetic resonance spectra were measured at 25° C. by using a Bruker Ultrashield Plus 400 MHz nuclear magnetic resonance instrument or a Bruker AVIII 600 MHz nuclear magnetic resonance instrument. High-resolution mass spectra were measured by using an Agilent 6220 electrospray ionization time-of-flight mass spectrometer. Optical rotation was measured by using a Schmidt & Haensch UniPol L 1000 automatic polarimeter at 589 nm, and the concentration (c) unit was g/100 mL. Infrared spectra were measured by using a Thermo Fisher Scientific Nicolet iS5 infrared instrument. Analytical high performance liquid chromatography was performed by using an Agilent 1200 series liquid phase chromatograph-quadrupole electrospray mass spectrometer 6130, and an analytical column used was a Thermo Scientific Hypercarb column (150×4.6 mm). Preparative high performance liquid chromatography was performed by using an Agilent 1200 series liquid phase chromatograph-quadrupole electrospray mass spectrometer 6130, a semi-preparative column used was a Thermo Scientific Hypercarb column (150×10 mm).

### Example 1

### synthesis of N-benzyl-N-benzyloxycarbonyl-5-aminopentyl 2-azido-3-O-acetyl-4-O-benzyl-2-deoxy-α-D-quinovopyranose (1*)

A reaction equation was as shown in FIG. 6.

Under the protection of nitrogen, 2-azido-3-O-acetyl-4-O-benzyl-2-deoxy-α-D-quinovopyranose trichloroacetimidate (C. Qin, J. Am. Chem. Soc. 2018, 140, 3120-3127) (1.13 g, 2.29 mmol) and N-benzyl-N-benzyloxycarbonyl-5-aminopentan-1-ol (B. Schumann, 2014, 5, 1992-2002) (1.13 g, 3.44 mmol) were dissolved in anhydrous ether/anhydrous dichloromethane mixed solution (57 mL, 3:1, v/v), activated molecular sieves (Aw-300 type) were added, and stirring was performed for 30 min. After the temperature of the reaction solution was decreased to −40° C., trimethylsilyl trifluoromethanesulfonate (0.63 mL, 3.44 mmol) was dropped slowly, and the reaction solution was stirred at this temperature until the reaction was completed. After the reaction was completed, triethylamine was dropped to quench the reaction, filtration was performed to remove the molecular sieves, and then distillation under reduced pressure was performed to remove the solvent. The obtained crude product was purified by silica gel column chromatography (petroleum ether:ethyl acetate, 15:1, v/v) to obtain a product 1* (1.31 g, 2.07 mmol, 90%, α/β=2.2:1). IR νₘₐₓ (film) 3030, 2934, 2105, 1749, 1694, 1506, 1496, 1453, 1421, 1362, 1222, 1067, 1044, 898, 767, 733, 697 cm⁻¹; ¹H NMR (400 MHz, CDCl₃) δ=7.41-7.14 (m, 13H, α-Ph-H, β-Ph-H), 5.51 (t, J=10.7 Hz, 0.5H, α3-H), 5.18 (d, J=12.3 Hz, 1.6H, α-PhCH₂, β-PhCH₂), 5.00 (t, J=10.4 Hz, 0.3H, β3-H), 4.84 (s, 1H, α1-H), 4.64 (s, 1.1H, α-PhCH₂), 4.60 (d, J=5.50 Hz, 0.4H, β-PhCH₂), 4.50 (s, 1.6H, α-PhCH₂, β-PhCH₂), 4.31 (s, 0.3H, β1-H), 3.93-3.55 (m, 1.3H, Linker-OCHₐ, α5-H), 3.50-3.30 (m, 1.1H, Linker-NCH₂, linker-OCH_{b}, β5-H, β2-H), 3.21 (m, 2.4H, α4-H, β4-H), 3.06 (dd, J=10.6, 3.5 Hz, 0.5H, α2-H), 2.05 (d, 2.4H, β-CH₃CO, α-CH₃CO), 1.58 (m, 3.2H, Linker-CH₂), 1.33 (d, J=6.2 Hz, 1H, β6-CH₃), 1.29 (d, J=6.3 Hz, 2.2H, α6-CH₃); ¹³C NMR (100 MHZ, CDCl₃) δ=170.1, 163.7, 156.8, 156.3, 137.8, 136.9, 128.6, 128.1, 127.9, 127.8, 127.1, 127.4, 101.9 (B anomeric), 97.9 (a anomeric), 92.0, 82.4, 81.6, 77.2, 75.0, 73.9, 72.0, 71.4, 70.1, 67.3, 66.8, 64.6, 61.6, 50.6, 50.3, 47.1, 46.3, 29.3, 27.9, 21.0, 17.9; HR-ESI-MS (m/z): calcd for C₃₅H₄₂N₄O₇Na⁺ (M+Na⁺): 653.1752, found: 653.1738.

### Example 2

### synthesis of N-benzyl-N-benzyloxycarbonyl-5-aminopentyl 2-acetamido-3-O-acetyl-4-O-benzyl-2-deoxy-α-D-quinovopyranose (2a*)

A reaction equation was as shown in FIG. 6.

At 0° C., thioacetic acid (22 mL) was added to anhydrous pyridine (22 mL) solution of the compound 1* (0.81 g, 1.31 mmol), the reaction solution was placed at room temperature, and stirring was performed overnight. After the reaction was completed, the reaction solution was azeotroped with toluene to remove the solvent, and the obtained crude product was purified by silica gel column chromatography (petroleum ether:acetone, 6:1, v/v) to obtain a colorless paste-like product 2a* (0.75 g, 1.49 mmol, 88%). [α]_{D}²⁰=+54.2° (c=1.00, CHCl₃); IR νₘₐₓ (film) 3324, 3030, 2933, 1738, 1693, 1518, 1496, 1453, 1422, 1363, 1304, 1233, 1134, 1073, 1046, 905, 735, 698 cm⁻¹; ¹H NMR (400 MHZ, CDCl₃) δ=7.43-7.14 (m, 15H, Ph-H), 5.89-5.62 (d, J=8.9 Hz, 1H, NH), 5.29-5.11 (m, 3H, 3-H, PhCH₂), 4.65 (m, 3H, 1-H, PhCH₂), 4.50 (s, 2H, PhCH₂), 4.22 (t, J=10.2, 3.5 Hz, 1H, 2-H), 3.76 (m, 1H, 5-H), 3.58 (m, 1H, Linker-CHₐ), 3.27 (m, 4H, 4-H, Linker-CH₂, linker-CH_{b}), 1.98 (s, 3H, CH₃CO), 1.92 (s, 3H, CH₃CO), 1.52 (m, 4H, Linker-CH₂), 1.28 (m, 5H, 6-CH₃, Linker-CH₂). ¹³C NMR (100 MHZ, CDCl₃) δ=171.3, 170.0, 156.7, 156.2, 137.8, 136.8, 128.5, 128.4, 127.9, 127.3, 97.0 (anomeric), 81.8, 77.4, 77.2, 77.0, 76.7, 75.1, 73.8, 67.8, 67.2, 66.9, 53.4, 52.6, 50.5, 50.2, 47.0, 46.1, 29.0, 27.9, 27.4, 23.5, 23.2, 21.0, 17.8; HR-ESI-MS (m/z): calcd for C₃₇H₄₆N₂O₈Na⁺ (M+Na⁺): 669.3152, found: 669.3144.

### Example 3

### synthesis of N-benzyl-N-benzyloxycarbonyl-5-aminopentyl 2-acetamido-4-O-benzyl-2-deoxy-α-D-quinovopyranose (3*)

A reaction equation was as shown in FIG. 6.

Sodium methoxide (0.03 g, 0.49 mmol) was added to methanol (33 mL) solution of the compound 2a* (0.64 g, 0.99 mmol), and the obtained reaction solution was stirred at room temperature. After the reaction was completed, the reaction solution was neutralized with an Amberlite IR 120 cation exchange resin, and an organic phase obtained after filtration was removed through distillation under reduced pressure. The obtained crude product was purified by silica gel column chromatography (petroleum ether:acetone, 4:1, v/v) to obtain a colorless paste-like product 3* (0.69 g, 0.97 mmol, 98%). [α]_{D}²⁰=+30.1° (c=1.00, CHCl₃); IR νₘₐₓ (film) 3324, 3030, 2930, 1692, 1543, 1496, 1453, 1422, 1367, 1307, 1227, 1115, 1069, 1043, 847, 732, 697 cm⁻¹; ¹H NMR (400 MHZ, CDCl₃) δ=7.41-7.20 (m, 15H, Ph-H), 6.20 (d, J=8.5 Hz, 0.5H, NH), 5.84 (d, J=8.4 Hz, 0.5H, NH), 5.17 (d, J=12.5 Hz, 2H, PhCH₂), 4.93 (d, J=11.1 Hz, 1H, PhCH₂), 4.71 (d, J=11.1 Hz, 1H, PhCH₂), 4.68 (s, 1H, 1-H), 4.49 (d, J=11.0 Hz, 2H, PhCH₂), 4.09 (t, J=9.5 Hz, 1H, 2-H), 3.81 (t, J=9.7 Hz, 1H, 3-H), 3.72-3.48 (m, 2H, 5-H, Linker-CHₐ), 3.37-3.17 (m, 3H, Linker-CH_{b}, Linker-CH₂), 3.12 (t, J=9.1 Hz, 1H, 4-H), 3.03 (s, 1H, 3-OH), 2.03 (s, 3H, CH₃CO), 1.66-1.42 (m, 4H, Linker-CH₂), 1.28 (m, 5H, 6-CH₃, Linker-CH₂); ¹³C NMR (100 MHZ, CDCl₃) δ=172.2, 156.4, 138.5, 137.9, 136.8, 128.7, 128.6, 128.4, 128.3, 128.1, 127.9, 127.5, 127.3, 97.0, 84.5, 77.5, 77.4, 77.2, 76.8, 75.3, 74.8, 67.6, 66.8, 54.6, 50.6, 47.0, 46.2, 29.2, 28.0, 27.4, 23.7, 18.0; HR-ESI-MS (m/z): calcd for C₃₅H₄₄N₂O₇Na⁺ (M+Na⁺): 627.3046, found: 627.3052.

### Example 4

### synthesis of phenyl 2-azido-3-O-benzyl-4,6-O-benzylidene-2-deoxy-1-selen-α-L-galactopyranose (4*)

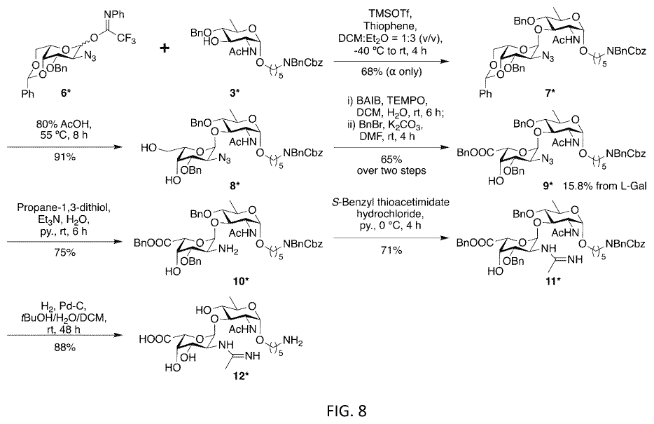

A reaction equation was as shown in FIG. 7.

Phenyl-2-azido-4,6-O-benzylidene-2-deoxy-1-selen-α-L-galactopyranose (Chemical synthesis of a synthetically useful L-galactosaminuronic acid building block. C. Qin, J. Chin. J. Nat. Med. 2022, 20, 387-392) (0.51 g, 1.18 mmol) was dissolved in anhydrous DMF (4 mL), sodium hydride (0.09 g, 2.36 mmol) was added, and stirring was performed overnight at room temperature. After the reaction was completed, the reaction solution was diluted with ethyl acetate, extraction was performed respectively with water and saturated saline solution, an obtained organic phase was dehydrated with anhydrous sodium sulfate, and then concentration was performed. The obtained crude product was purified by silica gel column chromatography (petroleum ether:ethyl acetate, 10:1, v/v) to obtain a white solid product 4* (0.54 g, 1.03 mmol, 87%). [α]_{D}²⁰=−132.1° (c=1.00, CHCl₃); IR νₘₐₓ (film) 3062, 3038, 2864, 2111, 1454, 1362, 1312, 1248, 1213, 1161, 1092, 1083, 1022, 913, 845, 740, 696 cm⁻¹; ¹H NMR (400 MHz, CDCl₃) δ=7.68-7.21 (m, 15H, Ph-H), 6.06 (s, 1H, 1-H), 5.51 (s, 1H, PhCH), 4.78 (d, J=5.0 Hz, 2H, PhCH₂), 4.43 (dd, J=10.3, 5.2 Hz, 1H, 2-H), 4.25 (d, J=3.2 Hz, 1H, 4-H), 4.13 (d, J=11.8 Hz, 1H, 6-CHₐ), 4.04 (s, 1H, 5-H), 4.01 (d, J=2.2 Hz, 1H, 6-CH_{b}), 3.79 (dd, J=10.3, 3.4 Hz, 1H, 3-H); ¹³C NMR (100 MHz, CDCl₃) δ=137.7, 134.6, 134.0, 129.3, 129.2, 129.0, 128.7, 127.9, 126.3, 101.1, 100.9, 91.9, 85.7 (anomeric), 77.6, 77.5, 77.4, 76.9, 76.8, 75.6, 73.4, 72.7, 71.6, 69.6, 65.3, 60.0, 44.3; HR-ESI-MS (m/z): calcd for C₂₆H₂₅N₃O₄SeNa⁺ (M+Na⁺): 546.0908, found: 546.0920.

### Example 5

### synthesis of 2-azido-3-O-benzyl-4,6-O-benzylidene-2-deoxy-α-L-galactopyranose (5*)

A reaction equation was as shown in FIG. 7.

The compound 4* (0.52 g, 0.99 mmol) was dissolved in THF/water mixed solution (2.5 mL, 1:1, v/v), bromosuccinimide (0.29 g, 2.49 mmol) was added, then the reaction solution was stirred at room temperature for 6 h. After the reaction was completed, the reaction solution was diluted with dichloromethane, and an organic phase was extracted with 10% Na₂S₂O₃/IM NaHCO₃ mixed solution (1:1, v/v). Distillation under reduced pressure was performed to remove the solvent, and then the obtained crude product was purified by silica gel column chromatography (petroleum ether:ethyl acetate, 5:1, v/v) to obtain a colorless paste-like product 5* (0.38 g, 0.94 mmol, 95%). [α]_{D}²⁰=−112.7° (c=1.00, CHCl₃); IR νₘₐₓ (film), 2921, 2852, 2108, 1681, 1597, 1338, 1299, 1229, 1132, 1110, 1065, 738, 697 cm⁻¹; ¹H NMR (400 MHz, CDCl₃) δ=7.58-7.22 (m, 10H, Ph-H), 5.46 (s, 1H, PhCH), 5.35 (t, J=3.0 Hz, 1H, 1-H), 4.79-4.69 (s, 2H, PhCH₂), 4.19 (s, 1H, 6-CHₐ), 4.16 (s, 1H, 3-H), 4.02-3.93 (m, 3H, 6-CH₆, 2-H, 5-H), 3.76 (s, 1H, 4-H), 3.33 (s, 1H, 1-OH); ¹³C NMR (100 MHZ, CDCl₃) δ=137.8, 129.1, 128.6, 126.3, 101.0, 96.3, 92.6, 78.0, 77.4, 76.8, 74.5, 73.0, 72.3, 71.7, 69.4, 66.7, 63.6, 62.6, 59.4, 29.7; HR-ESI-MS (m/z): calcd for C₂₀H₂₁N₃O₅Na⁺ (M+Na⁺): 406.1379, found: 406.1388.

### Example 6

### synthesis of 2-azido-3-O-benzyl-4,6-O-benzylidene-2-deoxy-α-L-galactopyranose trifluoroacetimidate (6*)

A reaction equation was as shown in FIG. 7.

Under the protection of nitrogen, the compound 5* (0.09 g, 0.22 mmol) was dissolved in anhydrous dichloromethane (2.5 mL), potassium carbonate (0.25 g, 1.77 mmol) and N-phenyl trifluoroacetimidoyl chloride (0.27 mL, 1.78 mmol) were added, and stirring was performed overnight at room temperature. After the reaction was completed, filtration was performed to remove the solid, rinsing with dichloromethane was performed, and the crude product obtained after concentration was purified by silica gel column chromatography (petroleum ether:ethyl acetate, 5:1, v/v) to obtain a product 6* (0.15 g, 0.28 mmol, 79%). 1H NMR (400 MHZ, CDCl₃) δ=7.68-7.01 (m, 14H, Ph-H), 6.84 (d, J=7.7 Hz, 2H, Ph-H, 1-H), 5.49 (s, 1H, Ph-CH), 4.76 (s, 2H, Ph-CH₂), 4.31 (d, J=12.6 Hz, 1H, 3-H), 4.11 (s, 2H, 5-H, 6-CHₐ), 3.99 (d, J=12.7 Hz, 1H, 3-H), 3.58-3.16 (m, 2H, 4-H, 6-CH_{b}); ¹³C NMR (100 MHZ, CDCl₃) δ=143.4, 137.4, 129.2, 128.7, 128.6, 128.3, 128.1, 127.9, 126.3, 124.4, 119.3, 101.2, 101.1, 77.9, 77.3, 77.2, 77.0, 76.7, 72.0, 71.9, 68.8, 67.4, 61.0, 29.7; HR-ESI-MS (m/z): calcd for C₂₈H₂₅F₃N₄O₅Na⁺ (M+Na⁺): 577.1675, found: 577.1688.

### Example 7

### synthesis of N-benzyl-N-benzyloxycarbonyl-5-aminopentyl 2-acetamido-4-O-benzyl-3-O-(2-azido-3-O-benzyl-4,6-O-benzylidene-2-deoxy-α-L-galactopyranose)-2-deoxy-α-D-quinovopyranose (7*)

A reaction equation was as shown in FIG. 8.

Under the protection of nitrogen, the trifluoroacetimidate saccharide donor 6* (0.09 g, 0.16 mmol) and the receptor 3* (0.14 g, 0.23 mmol) were dissolved in anhydrous dichloromethane/anhydrous ether mixed solution (5.2 mL, 1:3, v/v). thiophene (0.15 mL, 0.19 mmol) and activated molecular sieves (AW-300 type) were added, and the obtained reaction solution was stirred for 30 min at room temperature. After the temperature was decreased to −40° C., trimethylsilyl trifluoromethanesulfonate (3 μL, 0.016 mmol) was added, and then the reaction temperature was gradually increased to room temperature. After it was confirmed through TLC that the reaction was completed, triethylamine was dropped at 0° C. to quench the reaction, and filtration was performed by using diatomite to remove the molecular sieves. An organic phase was extracted with saturated sodium bicarbonate solution, then the crude product obtained after concentration was purified by silica gel column chromatography (petroleum ether:acetone, 6:1-3:1, v/v) to obtain a target disaccharide 7* (0.11 g, 0.11 mmol, 68%, a only). [α]_{D}²⁰=−152.8° (c=1.00, CHCl₃); IR νₘₐₓ (film) 2917, 2849, 2110, 1680, 1540, 1496, 1454, 1423, 1362, 1229, 1130, 1072, 735, 698 cm⁻¹; ¹H NMR (400 MHZ, CDCl₃) δ=7.46-7.06 (m, 25H, Ph-H), 6.19-5.75 (d, 1H, NH), 5.35 (d, J=3.5 Hz, 1H, 1′-H), 5.24 (s, 1H, PhCH), 5.18-5.07 (m, 2H, PhCH₂), 4.66 (d, J=8.8 Hz, 2H, PhCH₂), 4.61 (d, J=3.4 Hz, 1H, 1-H), 4.53 (t, J=8.8 Hz, 2H, PhCH₂), 4.45 (t, J=5.5 Hz, 2H, PhCH₂), 4.29 (d, J=10.6 Hz, 1H, 2-H), 3.96-3.78 (m, 5H, 3-H; 2′-H; 3′-H; 4′-H; 6′-CHₐ), 3.70 (m, 2H, 5-H, 5′-H), 3.53 (s, 1H, Linker-CHₐ), 3.41-3.14 (m, 4H, Linker-CH_{b}, Linker-CH₂, 6′-CH_{b}), 3.11 (t, J=9.4 Hz, 1H, 4-H), 1.95 (d, J=9.6 Hz, 3H, CH₃CO), 1.63-1.36 (m, 5H, Linker-CH₂, 6-CH₃); ¹³C NMR (100 MHZ, CDCl₃) δ=138.0, 129.1, 128.7, 128.6, 128.3, 128.1, 128.0, 127.9, 127.5, 126.3, 100.9, 83.8, 77.4, 77.2, 76.9, 73.1, 71.4, 69.2, 67.4, 53.6, 32.0, 29.8, 29.5, 29.2, 24.9, 22.8, 18.2, 14.3; HR-ESI-MS (m/z): calcd for C₅₅H₆₃N₅O₁₁Na⁺ (M+Na⁺): 992.4422, found: 992.4439.

### Example 8

### synthesis of N-benzyl-N-benzyloxycarbonyl-5-aminopentyl 2-acetamido-4-O-benzyl-3-O-(2-azido-3-O-benzyl-2-deoxy-α-L-galactopyranose)-2-deoxy-α-D-quinovopyranose (8*)

A reaction equation was as shown in FIG. 8.

The compound 7* (0.10 g, 0.1 mmol) was added to 80% acetic acid aqueous solution (2.5 mL), the temperature was increased to 55° C. through heating, and stirring was performed until the reaction was completed. Distillation under reduced pressure was performed to remove the solvent, then purification was performed by silica gel column chromatography (dichloromethane:methanol, 60:1-40:1-30:1, v/v) to obtain white solid 4,6-dihydroxysaccharide 8* (0.08 g, 0.09 mmol, 91%). [α]_{D}²⁰=−57.0° (c=1.00, CHCl₃); IR νₘₐₓ (film) 3350, 2917, 2849, 2110, 1680, 1540, 1517, 1496, 1454, 1423, 1362, 1229, 1130, 1072, 735, 698 cm⁻¹; ¹H NMR (600 MHZ, CDCl₃) δ=7.55-7.11 (m, 20H, Ph-H), 6.20 (d, J=9.3 Hz, 0.5H, NH), 5.92 (d, J=9.1 Hz, 0.5H, NH), 5.32 (s, 1H, 1′-H), 5.17 (s, 2H, PhCH₂), 4.73-4.58 (m, 5H, 1-H, 2PhCH₂), 4.50 (t, J=10.1 Hz, 2H, PhCH₂), 4.32 (d, J=10.5 Hz, 1H, 2-H), 3.92 (m, 2H, 3-H, 5′-H), 3.86-3.55 (m, 5H, 5-H, 2′-H, 3′-H, 4′-H Linker-CHₐ), 3.54 (d, J=4.9 Hz, 2H, 6′-CH₂), 3.29 (m, 3H, Linker-CH_{b} Linker-CH₂), 3.17 (t, J=9.4 Hz, 1H, 4-H), 2.77 (m, 1H, 4′-OH), 1.97 (d, J=11.9 Hz, 3H, CH₃CO), 1.70 (s, 1H, 6′-OH), 1.55 (m, 4H, Linker-CH₂), 1.31 (m, 5H, Linker-CH₂, 6-CH₃); ¹³C NMR (100 MHz, CDCl₃) δ=170.0, 156.5, 137.9, 137.3, 136.8, 128.9, 128.7, 128.6, 128.5, 128.1, 127.9, 127.4, 98.2, 97.9, 97.4, 83.9, 77.4, 77.2, 76.9, 76.2, 75.9, 75.1, 72.1, 69.5, 67.9, 63.1, 60.0, 53.6, 50.4, 47.3, 46.1, 29.8, 29.2, 28.9, 28.0, 27.5, 23.9, 23.5, 18.2; HR-ESI-MS (m/z): calcd for C₄₈H₅₉N₅O₁₁Na⁺ (M+Na⁺): 904.4109, found: 904.4116.

### Example 9

### synthesis of N-benzyl-N-benzyloxycarbonyl-5-aminopentyl 2-acetamido-4-O-benzyl-3-O-(2-azido-3-O-benzyl-2-deoxy-α-L-galactopyranose benzyl aldoate)-2-deoxy-α-D-quinovopyranose (9*)

A reaction equation was as shown in FIG. 8.

At 0° C., water (1 mL), 2, 2, 6, 6-tetramethylpiperidine 1-oxyl (TEMPO) (1.5 mg, 8.6 μmol), and diacetoxyliodobenzene (BAIB) (42 mg, 1.3 mmol) were added to dichloromethane solution (1 mL) of the compound 8* (38 mg, 43 μmol). After the reaction solution was stirred at room temperature for 4 h, the crude product obtained after silica gel column chromatography and concentration was directly inputted for the next reaction. The crude carboxylic acid compound was dissolved in anhydrous DMF (5 mL), sodium bicarbonate (14 mg, 0.1 mmol) and benzyl bromide (46 μL, 0.38 mmol) were sequentially added, and the obtained reaction solution was stirred at room temperature. After the reaction was completed, distillation under reduced pressure was performed to remove the solvent, and the obtained crude product was purified by silica gel column chromatography (petroleum ether:ethyl acetate, 5:1, v/v) to obtain yellow syrup-like benzyl glucuronate 9* (28 mg, 28 μmol, two-step reaction yield: 65%). [α]_{D}²⁰=−42.5° (c=1.00, CHCl₃); IR νₘₐₓ (film) 2957, 2920, 2849, 2113, 1721, 1500, 1457, 1360, 1213, 1161, 1094, 1073, 1028, 907, 740, 700 cm⁻¹; ¹H NMR (400 MHZ, CDCl₃) δ=7.41-7.15 (m, 25H, Ph-H), 5.98 (d, J=9.4 Hz, 1H, NH), 5.43 (s, 1H, 1′-H), 5.20 (d, J=12.3 Hz, 2H, PhCH₂), 5.16 (s, 1H, PhCHₐ), 4.98 (d, J=12.4 Hz, 1H, PhCHₐ), 4.72-4.69 (m, 2H, PhCHₐ, 5′-H), 4.68 (d, J=5.1 Hz, 1H, PhCHₐ), 4.63 (d, J=5.4 Hz, 2H, PhCH), 1-H), 4.55 (d, J=10.7 Hz, 1H, PhCH₆), 4.50 (d, J=5.4 Hz, 2H, NPhCH₂), 4.30 (t, J=11.7 Hz, 1H, 2-H), 4.16 (s, 1H, 4′-H), 3.93 (t, J=8.6 Hz, 1H, 3-H), 3.85 (dd, J=10.3, 3.1 Hz, 1H, 3′-H), 3.72 (m, 2H, 2′-H, 5-H), 3.55 (d, J=20.5 Hz, 1H, Linker-CHₐ), 3.37-3.19 (m, 3H, Linker-CH₂, Linker-CH₆), 3.16 (t, J=9.3 Hz, 1H, 4-H), 2.36 (s, 1H, 4′-OH), 1.97 (d, J=7.6 Hz, 3H, CH₃CO), 1.53 (m, 4H, Linker-CH₂), 1.32-1.24 (m, 5H, 6-CH₃, Linker-CH₂); ¹³C NMR (100 MHz, CDCl₃) δ=168.1, 137.9, 137.5, 137.0, 135.3, 128.7, 128.6, 128.5, 128.2, 128.0, 127.8, 127.3, 97.7, 97.2, 83.8, 77.2, 76.8, 76.3, 75.5, 75.1, 72.3, 70.3, 67.8, 67.2, 59.5, 53.3, 50.3, 47.1, 46.1, 29.0, 27.8, 27.4, 23.5, 18.0; HR-ESI-MS (m/z): calcd for C₅₅H₆₃N₅O₁₂Na⁺ (M+Na⁺): 1008.4371, found: 1008.4366.

### Example 10

### synthesis of N-benzyl-N-benzyloxycarbonyl-5-aminopentyl 2-acetamido-4-O-benzyl-3-O-(2-amino-3-O-benzyl-2-deoxy-α-L-galactopyranose benzyl aldoate)-2-deoxy-α-D-quinovopyranose (10*)

A reaction equation was as shown in FIG. 8.

Under the protection of nitrogen, the compound 9* (25 mg, 25.4 μmol) was dissolved in pyridine (2.6 mL), then water (0.24 mL, 13.2 mmol), triethylamine (0.05 mL, 0.4 mmol), and 1,3-propanediol (0.05 mL, 0.5 mmol) were added, the reaction solution was stirred at room temperature for 6 h until it was confirmed through TLC that the reaction of the raw materials was completed, distillation under reduced pressure was performed to remove he solvent, and the obtained crude product was purified by silica gel column chromatography (petroleum ether:acetone, 5:1-3:1, v/v) to obtain a colorless paste-like amino compound 10* (18.5 mg, 19.0 μmol, 75%). [α]_{D}²⁰=−57.1° (c=1.00, CHCl₃); IR νₘₐₓ (film) 3405, 3033, 2922, 1700, 1598, 1500, 1469, 1360, 1280, 1096, 1025, 741, 698 cm⁻¹; ¹H NMR (600 MHZ, CDCl₃) δ=7.39-7.13 (m, 25H, Ph-H), 6.70 (s, 0.5H, NH), 6.58 (s, 0.5H, NH), 5.30 (s, 1H, 1′-H), 5.22-5.12 (m, 3H, Ph-CH₂, PhCHₐ), 4.87 (d, J=12.5 Hz, 1H, PhCH_{b}), 4.69 (d, J=1.8 Hz, 1H, 5′-H), 4.68-4.62 (m, 2H, PhCHₐ, PhCHₐ), 4.60-4.53 (m, 2H, PhCH_{b}, PhCH_{b}), 4.50 (d, J=10.3 Hz, 2H, NPhCH₂), 4.22 (s, 1H, 2-H), 4.14 (t, J=2.4 Hz, 1H, 4′-H), 4.00 (d, J=10.5 Hz, 1H, 3-H), 3.73 (d, J=15.2 Hz, 1H, 5-H), 3.57 (s, 1H, 3′-H), 3.52 (s, 1H, Linker-CHₐ), 3.26 (s, 1H, Linker-CH₆), 3.23-3.18 (m, 3H, 2′-H, Linker-CH₂), 3.15 (t, J=9.4 Hz, 1H, 4-H), 1.94 (d, J=15.3 Hz, 3H, CH₃CO), 1.58-1.45 (m, 4H, Linker-CH₂), 1.26 (m, 5H, Linker-CH₂, 6-CH₃); ¹³C NMR (150 MHz, CDCl₃) δ=168.7, 137.8, 137.5, 135.4, 128.7, 128.5, 128.4, 128.2, 128.1, 128.0, 127.9, 127.8, 127.5, 127.3, 127.2, 99.5, 97.2, 83.6, 77.9, 77.2, 77.0, 76.8, 75.3, 74.9, 74.6, 71.8, 70.6, 67.8, 67.2, 67.1, 66.8, 66.6, 53.9, 50.2, 50.1, 47.1, 46.0, 31.9, 29.6, 29.3, 29.3, 29.2, 27.4, 23.5, 22.7, 18.0, 14.1; HR-ESI-MS (m/z): calcd for C₅₅H₆₅N₃O₁₂Na⁺ (M+Na⁺): 982.4466, found: 982.4450.

### Example 11

### synthesis of N-benzyl-N-benzyloxycarbonyl-5-aminopentyl 2-acetamido-4-O-benzyl-3-O-(2-acetamidino-3-O-benzyl-2-deoxy-α-L-galactopyranose benzyl aldoate)-2-deoxy-α-D-quinovopyranose (11*)

A reaction equation was as shown in FIG. 8.

Under the protection of argon, the amino compound 10* (18 mg, 18.74 μmol) was dissolved in 1 mL of anhydrous pyridine., the temperature was decreased to 0° C., and then benzyl thioacetimidate hydrochloride (5.7 mg, 28.12 μmol) was added. The reaction solution was stirred at 0° C. for 5 h, and the crude product obtained after concentration was purified by silica gel column chromatography (dichloromethane:methanol, 20:1, v/v) to obtain a colorless paste-like compound 11* (13.4 mg, 13.3 μmol, 71%). [α]_{D}²⁰=−80.2° (c=0.50, CHCl₃); IR νₘₐₓ (film) 3368, 3062, 2918, 1748, 1690, 1649, 1542, 1496, 1454, 1422, 1385, 1309, 1218, 1136, 1063, 911, 814, 735, 697, cm⁻¹; ¹H NMR (600 MHZ, CDCl₃) δ=7.51-6.95 (m, 25H, Ph-H), 6.77 (s, 1H, 2-NH), 5.46 (s, 1H, 1′-H), 5.16 (m, 2H, PhCH₂), 5.08 (d, J=12.3 Hz, 1H, PhCHₐ), 4.79 (d, J=12.4 Hz, 1H, PhCHₐ), 4.69 (d, J=11.6 Hz, 1H, PhCH₆), 4.64 (s, 1H, 5′-H), 4.58 (d, J=11.3 Hz, 1H, PhCHₐ), 4.54 (s, 1H, 1-H), 4.49 (m, 3H, Ph-CH₂, PhCH₆), 4.44 (d, J=11.3 Hz, 1H, PhCH_{b}), 4.26 (td, J=9.8, 3.6 Hz, 1H, 2-H), 4.17 (s, 1H, 4′-H), 4.05 (t, J=10.3 Hz, 1H, 3-H), 3.83 (s, 1H, 2′-H), 3.76 (m, 2H, 5-H, 3′-H), 3.52 (m, 1H, Linker-CHₐ), 3.29 (m, 3H, Linker-CH₂, Linker-CH₆), 3.16 (d, J=9.3 Hz, 1H, 4-H), 2.33 (s, 3H, Am-CH₃), 1.97 (s, 3H, CH₃CO), 1.55 (q, J=13.3 Hz, 4H, Linker-CH₂), 1.25 (d, J=6.6 Hz, 5H, Linker-CH₂, 6-CH₃); ¹³C NMR (150 MHz, CDCl₃) δ=166.5, 137.9, 137.3, 137.1, 128.6, 128.5, 128.4, 128.2, 128.1, 128.0, 127.9, 127.2, 97.6, 82.8, 77.2, 77.0, 76.8, 74.3, 71.0, 70.9, 70.1, 68.0, 67.2, 67.0, 66.6, 53.8, 51.6, 29.7, 29.0, 19.7, 17.9; HR-ESI-MS (m/z): calcd for C₅₇H₆₈N₄O₁₂Na⁺ (M+Na⁺): 1023.4731, found: 1023.4724.

### Example 12

### synthesis of 5-aminopentyl 2-acetamido-3-O-(2-acetamidino-2-deoxy-α-L-galactopyranose aldehyde acid)-2-deoxy-α-D-quinovopyranose (12*)

A reaction equation was as shown in FIG. 8.

The disaccharide 11* (13 mg, 12.9 μmol) was dissolved in tert-butanol/water/dichloromethane mixed solution (4 mL, 5:2:1, v/v/v), nitrogen displacement was performed on the reaction system, then a 10% palladium/carbon hydrogenation catalyst was added, and nitrogen displacement was continuously performed for 5 min. Hydrogen displacement was further performed on the reaction system for 5 min, then the reaction solution was stirred in a hydrogen environment for 48 h, the crude product obtained after filtration by using diatomite and concentration was preliminarily purified by a C₁₈ column (Macherey-Nagel, Diiren, Germany) (eluent was water and methanol), and the product was further purified by reverse-phase high performance liquid chromatography (semi-preparative Thermo Scientific Hypercarb column) to obtain a white solid target product 12* (5.8 mg, 11.4 μmol, 88%). [α]_{D}²⁰=−78.16° (c=0.10, H₂O); 1H NMR (400 MHZ, D₂O) δ=5.34 (d, J=3.8 Hz, 1H, 1′-H), 5.09 (d, J=1.4 Hz, 1H, 5′-H), 4.71 (d, J=3.7 Hz, 1H, 1-H), 4.40 (d, J=3.2 Hz, 1H, 4′-H), 4.16 (dd, J=10.5, 3.2 Hz, 1H, 3′-H), 4.11 (dd, J=10.3, 3.6 Hz, 1H, 2-H), 3.95 (dd, J=10.4, 3.8 Hz, 1H, 2′-H), 3.87 (m, 1H, 3-H), 3.79 (dd, J=9.8, 6.2 Hz, 1H, 5-H), 3.71-3.64 (m, 1H, Linker-CHₐ), 3.50-3.43 (m, 1H, Linker-CH₆), 3.31 (t, J=9.3 Hz, 1H, 4-H), 3.01 (t, J=7.7 Hz, 2H, Linker-CH₂), 2.29 (s, 3H, Am-CH₃), 2.01 (s, 3H, CH₃CO), 1.68 (dt, J=13.5, 7.0 Hz, 4H, Linker-CH₂), 1.47 (p, J=7.0 Hz, 2H, Linker-CH₂), 1.28 (d, J=6.2 Hz, 3H, 6-CH₃); ¹³C NMR (100 MHZ, D₂O) δ=173.7, 172.6, 166.3, 96.8, 95.5, 75.6, 73.8, 70.7, 69.0, 67.8, 67.7, 66.9, 53.8, 52.6, 39.5, 28.2, 26.6, 22.5, 22.0, 19.0, 16.7; HR-ESI-MS (m/z): calcd for C₂₁H₃₉N₄O₁₀⁺ (M+H⁺): 507.2661, found: 507.2645.

### Comparative Example 1

### synthesis of 6-(benzylthio) hexanol 2-azido-3-O-acetyl-4-O-benzyl-2-deoxy-α-D-quinovopyranose (13*)

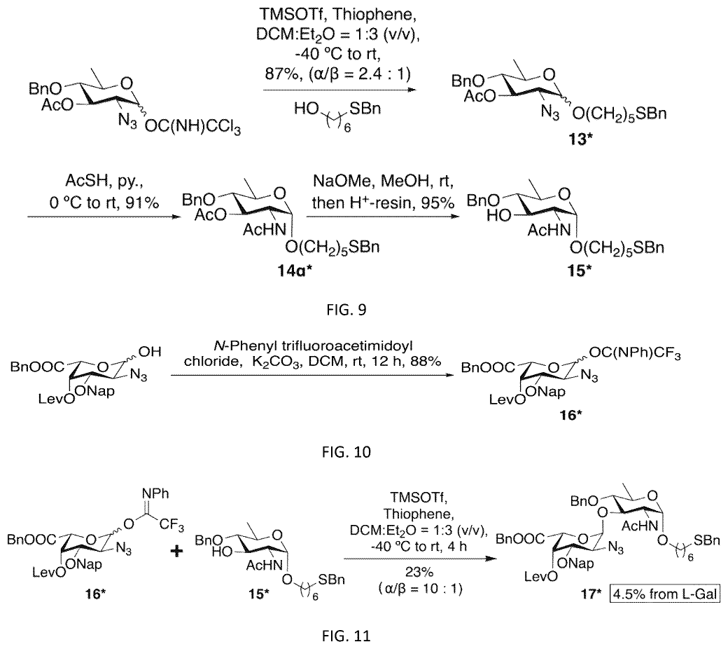

A reaction equation was as shown in FIG. 9.

Under the protection of nitrogen, 2-azido-3-O-acetyl-4-O-benzyl-2-deoxy-α-D-quinovopyranose trichloroacetimidate (C. Qin, J. Am. Chem. Soc. 2018, 140, 3120-3127) (1.25 g, 2.54 mmol) and 6-(benzylthio) hexanol (B. Schumann, 2014, 5, 1992-2002) (0.85 g, 3.81 mmol) were dissolved in anhydrous ether/anhydrous dichloromethane mixed solution (65 mL, 3:1, v/v), activated molecular sieves (Aw-300 type) were added, and stirring was performed for 30 min. After the temperature of the reaction solution was decreased to −40° C., trimethylsilyl trifluoromethanesulfonate (0.69 mL, 3.81 mmol) was dropped slowly, and the reaction solution was stirred at this temperature until the reaction was completed. After the reaction was completed, triethylamine was dropped to quench the reaction, filtration was performed to remove the molecular sieves, and then distillation under reduced pressure was performed to remove the solvent. The obtained crude product was purified by silica gel column chromatography (petroleum ether:ethyl acetate, 10:1, v/v) to obtain a product 13* (1.17 g, 2.20 mmol, 87%, α/β=2.4:1). IR νₘₐₓ (film) 3029, 2936, 2009, 1750, 1691, 1501, 1489, 1451, 1420, 1361, 1225, 1065, 1041, 892, 767, 732, 696 cm⁻¹; ¹H NMR (400 MHZ, CDCl₃) δ=7.45-7.10 (m, 12H, α-Ph-H, β-Ph-H), 5.52 (t, J=10.6 Hz, 0.5H, α3-H), 5.00 (t, J=10.5 Hz 0.3H, β3-H), 4.86 (d, J=3.5 Hz, 0.6H, α1-H), 4.63 (s, 1H, α-PhCH₂), 4.59 (d, J=3.4 Hz, 0.9H, β-PhCH₂), 4.33 (d, J=7.9 Hz, 0.3H, β1-H), 3.92-3.80 (m, 1H, α5-H), 3.69 (s, 2H, Linker-OCHₐ), 3.56-3.30 (m, 2H, Linker-SCH₂, Linker-OCH_{b}, β5-H, β2-H), 3.21 (m, 1H, α4-H), 3.05 (dd, J=10.6, 3.7 Hz, 0.8H, α2-H), 2.41 (t, J=7.3 Hz, 1H, β4-H), 2.04 (s, 2.9H, β-CH₃CO, α-CH₃CO), 1.55 (m, 7H, Linker-CH₂), 1.36 (m, 3H, Linker-CH₂), 1.33 (d, 6.1 Hz, 1H, β6-CH₃), 1.29 (d, J=6.2 Hz, 2.4H, α6-CH₃); HR-ESI-MS (m/z): calcd for C₂₈H₃₇N₃O₅SNa⁺ (M+Na⁺): 550.2352, found: 550.2339.

### Comparative Example 2

### synthesis of 6-(benzylthio) hexanol 2-acetamido-3-O-acetyl-4-O-benzyl-2-deoxy-α-D-quinovopyranose (14a*)

A reaction equation was as shown in FIG. 9.

At 0° C., thioacetic acid (37 mL) was added to anhydrous pyridine (37 mL) solution of the compound 13* (1.16 g, 2.20 mmol), the reaction solution was placed at room temperature, and stirring was performed overnight. After the reaction was completed, the reaction solution was azeotroped with toluene to remove the solvent, and the obtained crude product was purified by silica gel column chromatography (petroleum ether:acetone, 5:1, v/v) to obtain a colorless paste-like product 14a* (1.09 g, 2.00 mmol, 91%). [α]_{D}²⁰=+62.3° (c=1.00, CHCl₃); IR νₘₐₓ (film) 3325, 3031, 2937, 1740, 1699, 1520, 1495, 1450, 1423, 1361, 1301, 1236, 1130, 1075, 1046, 907, 733, 699 cm⁻¹; ¹H NMR (400 MHZ, CDCl₃) δ=7.42-7.13 (m, 10H, Ph-H), 5.71 (d, J=9.3 Hz, 1H, NH), 5.21 (dd, J=10.8, 9.2 Hz, 1H, 3-H), 4.69 (d, J=3.7 Hz, 1H, 1-H), 4.64 (s, 2H, PhCH₂), 4.31 (t, J=6.7 Hz, 0.5H, PhCH₂), 4.21 (m, 1H, 2-H), 3.78 (m, 1H, 5-H), 3.70 (s, 1.5H, PhCH₂), 3.62 (m, 1H, 4-H), 3.39-3.23 (m, 2H, Linker-CH₂), 2.41 (t, J=7.3 Hz, 2H, Linker-CH₂), 1.98 (s, 3H, CH₃CO), 1.92 (s, 3H, CH₃CO), 1.62-1.50 (m, 6H, Linker-CH₂), 1.28 (m, 5H, 6-CH₃, Linker-CH₂); ¹³C NMR (100 MHz, CDCl₃) δ=206.0 171.7, 166.3, 135.1, 134.5, 133.4, 133.3, 129.2, 128.8, 128.4, 128.1, 127.9, 127.4, 126.3, 126.2, 118.3, 100.8, 77.4, 77.1, 72.6, 72.1, 70.6, 67.9, 66., 62.4, 38.1, 29.8, 28.1; HR-ESI-MS (m/z): calcd for C₃₀H₄₁NO₆SNa⁺ (M+Na⁺): 566.7088, found: 566.7079.

### Comparative Example 3

### synthesis of 6-(benzylthio) hexanol 2-acetamido-4-O-benzyl-2-deoxy-α-D-quinovopyranose (15*)

A reaction equation was as shown in FIG. 9.

Sodium methoxide (0.04 g, 0.75 mmol) was added to methanol (50 mL) solution of the compound 14a* (0.81 g, 1.49 mmol), and the obtained reaction solution was stirred at room temperature. After the reaction was completed, the reaction solution was neutralized with an Amberlite IR 120 cation exchange resin, and an organic phase obtained after filtration was removed through distillation under reduced pressure. The obtained crude product was purified by silica gel column chromatography (petroleum ether:acetone, 4:1, v/v) to obtain a colorless paste-like product 15* (0.71 g, 1.42 mmol, 95%). [α]_{D}²⁰=+29.3° (c=1.00, CHCl₃); IR νₘₐₓ (film) 3328, 3025, 2931, 1688, 1540, 1492, 1457, 1421, 1360, 1305, 1223, 1117, 1066, 1043, 849, 730, 699 cm⁻¹; ¹H NMR (400 MHZ, CDCl₃) δ=7.41-7.06 (m, 10H, Ph-H), 5.81 (d, J=8.6 Hz, 1H, NH), 4.89 (d, J=11.1 Hz, 1H, PhCH₂), 4.68 (d, J=11.2 Hz, 1H, PhCH₂), 4.65 (d, J=3.9 Hz, 1H, 1-H), 4.05 (m, 1H, 2-H), 3.76 (t, J=9.2 Hz, 1H, 3-H), 3.69-3.58 (m, 3H, PhCH₂, Linker-CHₐ), 3.32 (m, 1H, Linker-CH₆), 3.08 (t, J=9.1 Hz, 1H, 4-H), 2.54 (s, 1H, 3-OH), 2.38 (m, 2H, Linker-CH₂), 2.00 (s, 3H, CH₃CO), 1.52 (m, 4H, Linker-CH₂), 1.31-1.15 (m, 7H, 6-CH₃, Linker-CH₂); HR-ESI-MS (m/z): calcd for C₂₈H₃₉NOSNa⁺ (M+Na⁺): 524.6718, found: 524.6724.

### Comparative Example 4

### synthesis of 2-azido-3-O-2-naphthylmethyl-4-O-levulinyl-2-deoxy-α-L-galactopyranose benzyl aldoate-trifluoroacetimidate (16*)

A reaction equation was as shown in FIG. 10.

Under the protection of nitrogen, 2-azido-3-O-2-naphthylmethyl-4-O-levulinyl-2-deoxy-L-galactopyranose benzyl aldoate (Chemical synthesis of a synthetically useful L-galactosaminuronic acid building block. C. Qin, Chin. J. Nat. Med. 2022, 20, 387-392) (10.7 mg, 0.02 mmol) was dissolved in anhydrous dichloromethane (0.5 mL), potassium carbonate (21.6 mg, 1.56 mmol) and N-phenyl trifluoroacetimidoyl chloride (23.3 μL, 1.56 mmol) were added, and then stirring was performed overnight at room temperature. After the reaction was completed, filtration was performed to remove the solid, rinsing with dichloromethane was performed, and the crude product obtained after concentration was purified by silica gel column chromatography (petroleum ether:ethyl acetate, 5:1, v/v) to obtain a product 16* (10.1 mg, 0.014 mmol, 88%). 1H NMR (400 MHZ, CDCl₃) δ=7.90-6.79 (m, 17H, Ph-H), 5.84 (s, 1H, 4-H), 5.34-5.14 (m, 2H, PhCH₂), 4.91 (d, J=11.5 Hz, 1H, PhCH₂), 4.67 (d, J=11.5 Hz, 1H, PhCH₂), 4.12 (d, J=7.2 Hz, 1H, 5-H), 3.88 (t, J=9.2 Hz, 1H, 2-H), 3.53 (d, J=9.4 Hz, 1H, 3-H), 2.75-2.48 (m, 4H, Lev-CH₂), 2.12 (s, 3H, CH₃CO); ¹³C NMR (100 MHZ, CDCl₃) δ=206.0, 171.5, 165.4, 134.9, 134.1, 133.3, 129.3, 128.9, 128.8, 128.5, 128.1, 127.9, 127.6, 126.4, 126.3, 126.2, 124.6, 119.4, 77.4, 77.1, 73.3, 72.2, 68.1, 66.3, 61.3, 38.0, 29.8, 28.0; HR-ESI-MS (m/z): calcd for C₃₇H₃₃F₃N₄O₈N^{a}+ (M+Na⁺): 741.2148, found: 741.2155.

### Comparative Example 5

### synthesis of 6-(benzylthio) hexanol 2-acetamino-4-O-benzyl-3-O-(2-azido-3-O-2-naphthylmethyl-4-O-levulinyl-2-deoxy-α-L-galactopyranose benzyl aldoate)-2-deoxy-α-D-quinovopyranose (17*)

A reaction equation was as shown in FIG. 11.

Under the protection of nitrogen, the trifluoroacetimidate saccharide donor 16* (9 mg, 0.013 mmol) and the receptor 15* (14 mg, 0.028 mmol) were dissolved in anhydrous dichloromethane/anhydrous ether mixed solution (0.5 mL, 1:3, v/v). thiophene (15 μL, 0.15 mmol) and activated molecular sieves (AW-300 type) were added, and the obtained reaction solution was stirred for 30 min at room temperature. After the temperature was decreased to −40° C., trimethylsilyl trifluoromethanesulfonate (0.25 μL, 12.5 μmol) was added, and then the reaction temperature was gradually increased to room temperature. After it was confirmed through TLC that the reaction was completed, triethylamine was dropped at 0° C. to quench the reaction, and filtration was performed by using diatomite to remove the molecular sieves. An organic phase was extracted with saturated sodium bicarbonate solution, then the crude product obtained after concentration was purified by silica gel column chromatography (petroleum ether:acetone, 6:1-3:1, v/v) to obtain a target disaccharide 17* (3.1 mg, 0.003 mmol, 23%, α/β-10:1). [α]_{D}²⁰=−170.3° (c=0.50, CHCl₃); IR νₘₐₓ (film) 3329, 2930, 2110, 1740, 1677, 1375, 1230, 1040, 970, 750, 698 cm⁻¹; ¹H NMR (400 MHZ, CDCl₃) δ=7.85-7.10 (m, 22H, Ph-H), 5.86 (d, J=9.4 Hz, 1H, NH), 5.67 (dd, J=3.3, 1.8 Hz, 1H, 4′-H), 5.45 (d, J=3.4 Hz, 1H, 1′-H), 5.16 (d, J=11.9 Hz, 1H, PhCHₐ), 4.94 (d, J=12.0 Hz, 1H, PhCH₆), 4.86-4.79 (m, 2H, PhCHₐ, 5′-H), 4.66-4.50 (m, 4H, 1-H, PhCH_{b}, PhCH₂), 4.31 (td, J=9.9, 3.8 Hz, 1H, 2-H), 3.98-3.87 (m, 2H, 3′-H, 3-H), 3.79-3.68 (m, 3H, 5-H, SCH₂Ph), 3.68-3.54 (m, 2H, 2′-H, Linker-CHₐ), 3.33 (dt, J=9.8, 6.7 Hz, 1H, Linker-CH₆), 3.16 (t, J=9.3 Hz, 1H, 4-H), 2.63-2.30 (m, 6H, Lev-CH₂, Linker-CH₂), 2.02 (s, 3H, Lev-CH₃), 1.94 (s, 3H, CH₃CO), 1.57 (m, 4H, Linker-CH₂), 1.46-1.16 (m, 5H, 6-CH₃, Linker-CH₂). ¹³C NMR (100 MHz, CDCl₃) δ=171.4, 167.3, 137.6, 134.6, 133.4, 133.2, 129.2, 129.0, 128.7, 128.6, 128.4, 128.1, 127.8, 127.3, 127.0, 126.2, 97.8 (C1′), 97.3 (C1), 83.9, 77.5, 77.4, 77.2, 77.0, 76.8, 75.3, 73.9, 72.2, 69.6, 68.0, 67.7, 67.4, 59.8, 53.5, 38.1, 36.5, 31.4, 29.8, 29.4, 29.2, 28.7, 28.0, 26.0, 23.6, 18.2.; HR-ESI-MS (m/z): calcd for C₅₇H₆N₄O₁₂SNa⁺ (M+Na⁺): 1053.4296, found: 1053.4282.

Although disclosed with preferred examples above, the disclosure is not limited by the examples. Any person skilled in the art may make various alternations and modifications without departing the spirit and scope of the disclosure. Therefore, the scope of protection of the disclosure should be subject to the scope as defined in the claims.

What is claimed is:

1. A chemical synthesis method for a disaccharide fragment assembled with a linker of a Vibrio vulnificus biotype 2 serovar A O-antigen, wherein a chemical structural formula of the disaccharide fragment is as expressed by general formula I: 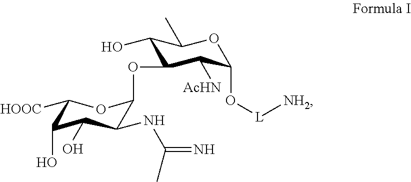
where a linker L is a chain structure with 2-40 carbon atoms and containing 0-6 heteroatoms, a substituted or unsubstituted three-membered to six-membered ring structure, an amide bond, or a carbamido group;
wherein the chemical synthesis method comprises using the following two monosaccharide building blocks 1 and 2 and a linker 3 as raw materials: 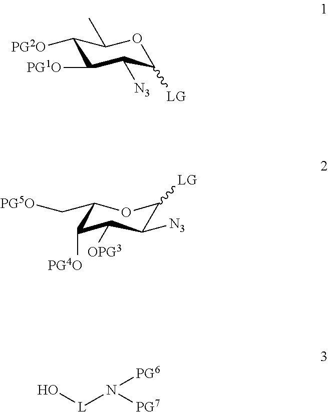
where
PG¹ is a hydroxyl protecting group selected from acetyl, benzoyl, levulinoyl, pivaloyl, allyloxycarbonyl, chloroacetyl, dichloroacetyl, trichloroacetyl, 2-naphthylmethyl, para-methoxybenzyl, tert-butyldimethylsilyl, tert-butyldiphenylsilyl, and triethylsilyl;
PG² is a hydroxyl protecting group selected from 2-naphthylmethyl and benzyl;
PG³ is a hydroxyl protecting group selected from 2-naphthylmethyl, benzyl, acetyl, levulinoyl, benzoyl, chloroacetyl, dichloroacetyl, trichloroacetyl, pivaloyl, allyloxycarbonyl, benzyl, 2-naphthylmethyl, para-methoxybenzyl, tert-butyldimethylsilyl, tert-butyldiphenylsilyl, and triethylsilyl;
PG⁴ and PG⁵ are hydroxyl protecting groups selected from benzylidene acetal and isopropylidene ketal;
PG⁶ and PG⁷ are amino protecting groups selected from benzyl and benzyloxycarbonyl; and
LG is a leaving group used for a glycosylation reaction selected from bromine, fluorine, ethylthio, para-tolylthio, phenylthio, trichloroacetimidate, N-phenyl trifluoroacetimidate, and dibutyl phosphate; and
the chemical synthesis method sequentially comprises the following steps:
reaction A: enabling the monosaccharide building block 1 to have a glycosidation reaction with the linker 3;
reaction B: after completing the glycosidation reaction, performing reduction and acetylation on an azido group at position 2 in the structure;
reaction C: after completing the reduction and acetylation of the azido group, performing deprotection on position 3 to remove the protecting group PG¹ to obtain a D-quinovosamine receptor used for assembling a disaccharide, and then enabling the D-quinovosamine receptor to experience a glycosidation reaction with the monosaccharide building block 2 to obtain an initial disaccharide;
reaction D: performing deprotection on positions 4 and 6 in the monosaccharide building block 2 in the obtained initial disaccharide to remove the protecting groups PG⁴ and PG⁵;
reaction E: after completing the deprotection, oxidizing a primary hydroxyl group at position 6 in the monosaccharide building block 2 to a carboxyl group;
reaction F: subsequently performing protection on the carboxyl group after oxidization;
reaction G: after the carboxyl group is protected, reducing the azido group at position 2 in the monosaccharide building block 2 to an amino group;
reaction H: after reducing to the amino group, constructing an acetamidino group through a modifying reagent; and finally
reaction I: performing global deprotection to remove the protecting group PG² in the monosaccharide building block 1, the protecting group of the carboxyl group and the protecting group PG³ in the monosaccharide building block 2, and the protecting groups PG⁶ and PG⁷ in the linker 3 to obtain the disaccharide fragment assembled with a linker of a V. vulnificus biotype 2 serovar A O-antigen as expressed by general formula I.

2. The chemical synthesis method according to claim 1, wherein a synthesis route is as follows: 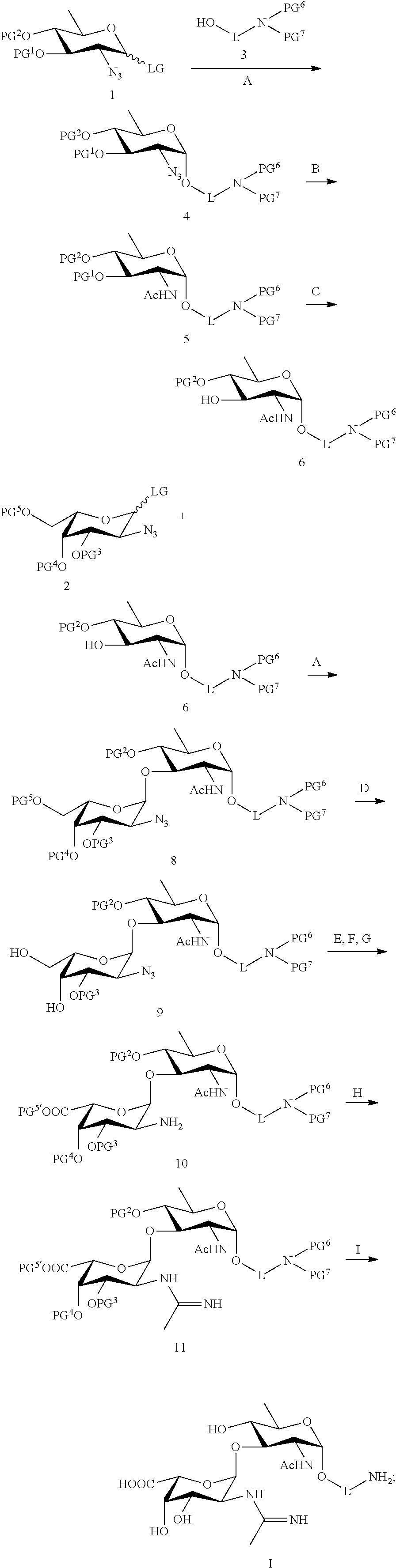
where
PG⁵′ is a protecting group of the carboxyl group selected from methyl, tert-butyl, allyl, triphenylmethyl, diphenylmethyl, tert-butyldimethylsilyl, propargyl, benzyl, para-methoxybenzyl.

3. The chemical synthesis method according to claim 1, wherein in reaction A, the glycosidation reaction is promoted by using an activator; and the activator is any one or more selected from methyl trifluoromethanesulfonate, dimethylmethylthiosulfonium trifluoromethanesulfonate, trifluoromethanesulfonic acid, and trimethylsilyl trifluoromethanesulfonate.

4. The chemical synthesis method according to claim 1, wherein in reaction A, the temperature for the glycosidation reaction ranges from −40° C. to room temperature.

5. The chemical synthesis method according to claim 1, wherein in reaction B, the reduction and acetylation of the azido group are completed directly by using thioacetic acid and pyridine; or the azido group is firstly reduced to the amino group and then acetylation is performed.

6. The chemical synthesis method according to claim 1, wherein in reaction E, the primary hydroxyl group is oxidized to the carboxyl group by using an oxidizing reagent; and the oxidizing reagent is any one or more of Jones reagent, Collins reagent, pyridinium chlorochromate, or pyridinium dichromate;
or the primary hydroxyl group is oxidized to the carboxyl group under the action of dimethyl sulfoxide and an activator; and the activator is any one or more of cyclohexyl carbodiimide, acetic anhydride, trifluoroacetic anhydride, phosphorus pentoxide, pyridine/sulfur trioxide, and oxalyl chloride.

7. The chemical synthesis method according to claim 1, wherein in reaction G, the azido group is reduced to the amino group by using any system of trimethylphosphine/water, triphenylphosphine/water, 1,3-dimercaptopropane/triethylamine, sodium borohydride/nickel dichloride, tin dichloride/phenylthiol/triethylamine, zinc/copper/acetic acid, and Lindlar catalyst/hydrogen.

8. The chemical synthesis method according to claim 1, wherein in reaction H, the amino group is modified to the acetamido group by using aryl thioacetimidate hydrohalide or alkyl thioacetimidate hydrohalide under treatment of an alkali;
or the amino group is modified to the acetamido group by using alkyl acetimidate hydrohalide under treatment of an alkali.

9. A disaccharide fragment assembled with a linker of a V. vulnificus biotype 2 serovar A O-antigen synthesized by the chemical synthesis method according to claim 1, wherein a chemical structural formula of the disaccharide fragment is general formula I: 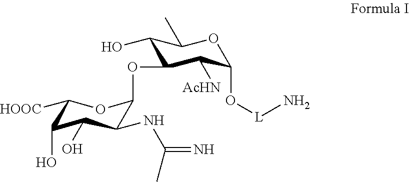
where a linker L is a chain structure with 2-40 carbon atoms and containing 0-6 heteroatoms, a substituted or unsubstituted three-membered to six-membered ring structure, an amide bond, or a carbamido group.

10. A V. vulnificus vaccine, containing the disaccharide fragment assembled with a linker of a V. vulnificus biotype 2 serovar A O-antigen according to claim 9.

11. A drug for treating a disease caused by V. vulnificus infection, containing the disaccharide fragment assembled with a linker of a V. vulnificus biotype 2 serovar A O-antigen according to claim 9.

12. A drug composition for treating Pseudomonas aeruginosa infection according to claim 11, further containing a pharmaceutical auxiliary material.
